(19) United States
(12) Patent Application Publication
(10) Pub. No.: US 2023/0270962 A1
(43) Pub. Date: Aug. 31, 2023

(54) ADAPTIVE HUMIDIFICATION IN HIGH FLOW NASAL THERAPY

(71) Applicant: KONINKLIJKE PHILIPS N.V., Eindhoven (NL)

(72) Inventors: Pascal de Graaf, Eindhoven (NL); Samer Bou Jawde, Cambridge, FL (US); Harold Johannes Antonius Brans, Eindhoven (NL)

(21) Appl. No.: 18/095,056

(22) Filed: Jan. 10, 2023

Related U.S. Application Data
(60) Provisional application No. 63/314,557, filed on Feb. 28, 2022.

(51) Int. Cl. A61M 16/00; A61M 16/06; A61M 16/16

(52) U.S. Cl. CPC A61M 16/024; A61M 16/0666; A61M 16/16; A61M 2016/0027

(57) ABSTRACT

According to an aspect, there is provided a cannula for use in high flow nasal therapy, comprising: a first tube for directing a first fluid from a nasal cavity of a subject to a location outside of the subject; a second tube for directing a second fluid from a supply of the second fluid to the nasal cavity of the subject; a flow sensor located within the first tube, the flow sensor configured to measure a flow rate of the first fluid moving through the first tube; and a humidity sensor located within the first tube, the humidity sensor configured to measure a humidity of the first fluid moving through the first tube; wherein the measured flow rate and the measured humidity are to be used by a processor to control a humidifier to adjust a humidity of the second fluid to be supplied to the subject.

### CROSS-REFERENCE TO RELATED APPLICATIONS

This patent application claims the priority benefit under 35 U.S.C. § 119(e) of U.S. Provisional Application No. 63/314,557, filed on Feb. 28, 2022, the contents of which are herein incorporated by reference.

### FIELD OF THE INVENTION

The invention relates to an apparatus and method for monitoring parameters relating to high flow nasal therapy, and, more particularly, to monitoring a flow rate and a humidity of fluid moving through a tube from a nasal cavity of a subject to a location outside of the subject.

### BACKGROUND OF THE INVENTION

High flow nasal therapy (HFNT) is a therapy that may be used in a medical setting, such as a hospital, to treat hypoxemic patients, for example those suffering from a coronavirus infection. HFNT may be used to supply a fluid, such as oxygen, to a subject at a high flow rate (e.g., up to 60 litres per minute). Due to the high flow rate of supplied fluid, HFNT has the potential to dry out a subject's airways, and therefore the fluid supplied to the subject may be humidified. The high flow rates of the supplied fluid may demand a high power humidifier able to supply sufficient quantities of water vapor to a subject (e.g., the humidifier may use up to 200 millilitres of water per hour at 60 litres of gas per minute). Maintaining the water levels in the humidifier can put a strain on nursing staff. Additionally, HFNT may be used in other settings, such as the home, to treat hypercapnia. In a home setting, the burden of maintaining the humidifier is even heavier, as it can come down to the subject or informal caregiver to monitor and resupply the water level. Having a humidifier that minimizes water use while maintaining a sufficiently humidified airway would be a benefit in both of these settings.

### SUMMARY OF THE INVENTION

There is a desire for a device in which a fluid can be supplied to a subject such that the humidity of the fluid can be controlled, or optimized, so that the subject's physiological state is maintained at a stable level. More specifically, a subject receiving high flow nasal therapy can experience a drying out of their airways, which may be combatted by supplying a humidified fluid to the subject. To ensure that the subject's airways are properly humidified, the fluid may be humidified to a high degree (e.g., 100% relative humidity), which may lead to the subject experiencing discomfort. In addition, a fluid humidified to a high degree may require a high power humidifier, which may require topping up with water regularly to prevent the humidifier from running dry. Embodiments disclosed herein provide a solution to these problems, enabling a determination of a humidity state of a subject's airways, which can be used to optimize a humidity setting of a humidifier used to humidify a fluid supplied to the subject. Optimizing a humidity setting of a humidifier can reduce water usage in an informed way, based on feedback relating to the effect of the humidification on the subject, rather than relying solely on a pre-set humidity level. Reducing water usage by a humidifier used to humidity a fluid to be supplied to a subject also has the benefit of having to refill the humidifier less often, reducing condensation in tubing used to supply the fluid to the subject (which may be detrimental to the health of a subject), and reducing the overall humidity build up in the room where the subject is located.

According to a first specific aspect, there is provided a cannula for use in high flow nasal therapy, the cannula comprising a first tube for directing a first fluid from a nasal cavity of a subject to a location outside of the subject; a second tube for directing a second fluid from a supply of the second fluid to the nasal cavity of the subject; a flow sensor located within the first tube, the flow sensor configured to measure a flow rate of the first fluid moving through the first tube; and a humidity sensor located within the first tube, the humidity sensor configured to measure a humidity of the first fluid moving through the first tube; wherein the measured flow rate and the measured humidity are to be used by a processor to control a humidifier to adjust a humidity of the second fluid to be supplied to the subject.

In some embodiments, the first tube and the second tube may be arranged concentrically relative to one another and/or the second tube may be located within the first tube, wherein the flow sensor and the humidity sensor may be located between the second tube and the first tube.

The flow sensor may, in some embodiments, comprise a thin-film thermal flow sensor.

In some embodiments, the humidity sensor may comprise an integrated capacitive membrane sensor.

The cannula may, in some embodiments, further comprise a humidification connection to couple the cannula to a humidifier configured to adjust a humidity of the second fluid to be supplied to the subject.

According to a second specific aspect, there is provided a computer-implemented method comprising: receiving flow rate data, measured using a flow sensor, the flow rate data indicative of a flow rate of a first fluid moving through a tube for directing the first fluid from a nasal cavity of a subject to a location outside of the subject; receiving humidity data indicative of a humidity of the first fluid moving through the tube; determining, based on the flow rate data and/or the humidity data, a first time point at which the subject begins an exhalation; determining, based on the humidity data, a second time point during the exhalation at which the humidity of the first fluid reaches a defined humidity level; determining, based on the flow rate data, a volume of the first fluid passing the flow sensor from the first time point to the second time point; comparing the volume of the first fluid to a reference volume; and generating, based on the comparison, a signal to control a humidification setting of a humidifier such that the humidity of a second fluid to be supplied to the subject is updated.

In some embodiments, the computer-implemented method may further comprise: determining, at a third time point, a reference flow rate of the second fluid; and applying, based on the reference flow rate, a correction to the flow rate data to account for the flow rate of the second fluid.

The computer-implemented method may, in some embodiments, further comprise: determining, at a third time point, a reference humidity of the second fluid; and applying, based on the reference humidity, a correction to the humidity data to account for the humidity of the second fluid to be supplied to the subject.

In some embodiments, the computer-implemented method may further comprise: determining, based on the flow rate data and/or the humidity data, a fourth time point at which the subject begins to inhale; and generating a signal to control a flow rate of the second fluid, such that the second fluid to be supplied to the subject between the first time point and the fourth time point is reduced.

The computer-implemented method may, in some embodiments, further comprise: receiving a user preference to reduce or increase a level of humidity in the second fluid to be supplied to the subject; and updating, based on the user preference, the generated signal to control a humidification setting of a humidifier.

In some embodiments, the computer-implemented method may further comprise generating an alert signal in response to determining that oscillations in the flow rate data exceed a defined frequency and/or in response to determining that the humidity of the first fluid falls below a threshold level.

In some embodiments, the subject may receive a supply of the second fluid via a second tube, wherein the reference volume may be determined based on data obtained in the absence of the second fluid being supplied to the subject via the second tube.

The computer-implemented method may, in some embodiments, further comprise generating an alert signal in response to determining that the volume of the first fluid deviates from the reference volume by a defined amount.

In some embodiments, the defined humidity level may be in the range 30 to 44 mg/l H₂O at 37° C.

According to a third specific aspect, there is provided a computer program product comprising a non-transitory computer readable medium, the computer readable medium having computer readable code embodied therein, the computer readable code being configured such that, on execution by a suitable computer or processor, the computer or processor is caused to perform one or more steps of the methods disclosed herein.

### BRIEF DESCRIPTION OF THE DRAWINGS

Exemplary embodiments will now be described, by way of example only, with reference to the following drawings, in which:

FIG. 1 is an illustration of an example of a subject with a cannula;

FIG. 2A is a graph showing an example of how humidity of a fluid changes as a function of time during inhalation and exhalation of a subject;

FIG. 2B is a graph showing an example of how flow rate of a fluid changes as a function of time during inhalation and exhalation of a subject;

FIG. 3 is an illustration of an example of a cannula for use in high flow nasal therapy;

FIG. 4 is an illustration of an example of a cannula for use in high flow nasal therapy;

FIG. 5 is a flowchart of an example of a computer-implemented method for generating a signal to control a humidification setting of a humidifier;

FIG. 6 is a flowchart of a further example of a computer-implemented method for generating a signal to control a humidification setting of a humidifier; and

FIG. 7 is a schematic illustration of an example of a processor in communication with a computer-readable medium.

### DETAILED DESCRIPTION OF EMBODIMENTS

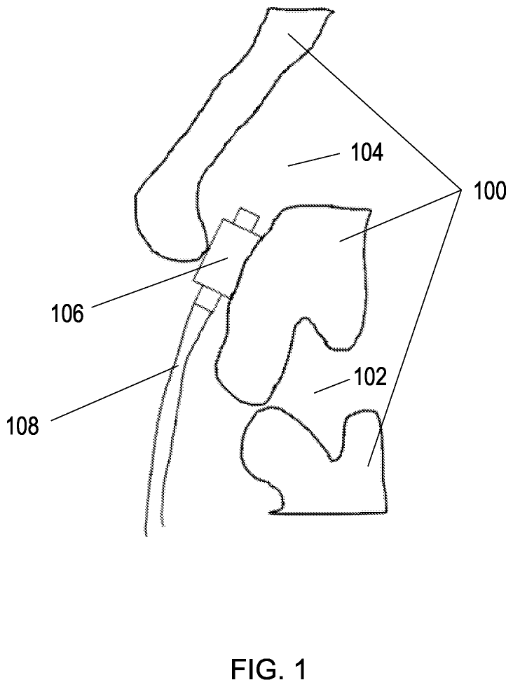

FIG. 1 is an illustration of an example of part of a subject 100, including a mouth 102 and a nasal cavity 104. The nasal cavity is a fluid-filled space (e.g., filled with air) behind the nose of the subject, which forms part of the subject's upper respiratory tract. A nasal septum usually divides the nasal cavity into two parts, or cavities, which may be referred to as a right nasal cavity and a left nasal cavity). In some examples, the nasal cavity 104 may refer to the two cavities combined while in other examples the nasal cavity 104 may refer to only one of these cavities (e.g., the right nasal cavity or the left nasal cavity). FIG. 1 also shows a cannula 106 used to supply a fluid, such as oxygen, to the subject 100 via a tube 108. In some examples, the cannula 106 may comprise or include the tube 108.

A fluid (e.g., air, oxygen, or the like) in a subject's alveoli may be saturated with water vapour to enable efficient gas exchange. As the subject inhales, fluid (e.g., air, oxygen, or the like) entering their lungs may be heated and humidified by their upper airways (e.g., by their airway lining). This means that, starting from the point where the fluid enters the subject's body (e.g., the subject's nose), the fluid may increase in temperature and humidity until it reaches body temperature and pressure (e.g., atmospheric pressure), and is saturated with water vapour. In other words, inhaled fluid may be heated and humidified until the temperature and the humidity of the fluid become similar to (e.g., equal to) the temperature and humidity of any fluid in the subject's alveoli (e.g., 44 mg/l H₂O at 37° C.). At this point, the inhaled fluid may be said to be at BTPS conditions (body temperature and pressure, saturated). The anatomical point at which the inhaled fluid reaches BTPS conditions may be referred to as an Isothermic Saturation Boundary (ISB), which may be below a subject's upper airways (e.g., 5 cm below their carina). When the subject's upper airways are exposed to a higher flow rate of fluid, for example due to physical exertion resulting in panting or due to mechanical ventilation, the airway tissue may no longer be able to supply the required heat and moisture to the fluid, which can lead to the airway drying out and possible discomfort. In these circumstances, the ISB may start to shift downwards (e.g., further into the airway and/or lungs of the subject) to allow more time for the airway of the subject to heat and humidify the fluid. In the invention disclosed herein, the location of the ISB may be used as an indicator of the humidification state of the lungs of a subject.

A humidity value of a fluid expressed in units of milligrams of H₂O per litre (e.g., mg/l H₂O) refers to an absolute humidity of the fluid. A fluid having an absolute humidity of 44 mg/l H₂O at a temperature of 37° C. may correspond to a relative humidity of approximately 100%. As the temperature of the fluid increases, the maximum absolute humidity of the fluid may increase, and vice versa. When a fluid is at a maximum absolute humidity, it may be said to be at 100% relative humidity.

For a subject receiving ventilation (e.g., mechanical ventilation), fluid supplied to the subject may be humidified to avoid drying out the lining of the subject's airway. The ability to humidify inhaled fluid can differ between subjects. As a result, a relatively high level of humidification may be used (e.g., 33 to 44 mg/l H₂O), which may prevent the subject's airways from drying out, but can be uncomfortable (e.g., the subject may experience this level of humidity as hot and stuffy). Both under and over humidification may lead to complications and thus an intended (e.g., optimal) humidification level would be preferable. Embodiments disclosed herein relate to determining a humidification level of a fluid to be supplied to a subject that takes account of the natural humidification ability of the subject's airway. This may be achieved by modifying a level of humidity of a fluid to be supplied to the subject such that the location of their ISB is the same, or similar, for ventilation as for unsupported (normal) breathing.

The location of a subject's ISB may be determined by analysing the humidity, and flow rate, of exhaled fluid as a function of time. The humidity of the exhaled fluid may provide information relating to the humidity of subject's airways. The humidity of the exhaled fluid may be associated with a corresponding exhaled portion of the tidal volume by measuring the flow rate of the exhaled fluid.

In some examples, the term “fluid” may refer to a fluid moving in a direction from a nasal cavity of a subject to a location outside of the subject, which, in turn, may be referred to as a first fluid, an exhaled fluid, an exhalation fluid, or the like. The first fluid may comprise air, oxygen, saliva, a combination of fluids, or the like. In some examples, a fluid may refer to a fluid being supplied, from a fluid supply, to a nasal cavity of the subject, which, in turn, may be referred to as a second fluid, inhaled fluid, inhalation fluid, or the like. A second fluid may comprise air, oxygen, or the like.

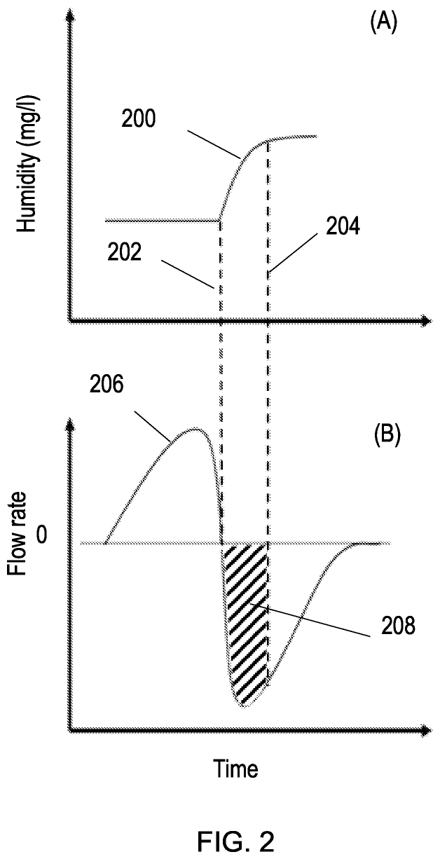

FIG. 2A is a graph showing an example of how the humidity of a fluid may change as a function of time during inhalation and exhalation of a subject, as represented by line 200. The x-axis represents time and the y-axis represents humidity. The humidity level of the fluid remains constant until a time indicated by dashed line 202, at which point the humidity of the fluid starts to increase. In this example, the subject may be inhaling up to the time point indicated by line 202, wherein the humidity may correspond to that of the ambient air that the subject is inhaling and/or a fluid being supplied to the subject (e.g., oxygen). The humidity begins to rise at the time point represented by line 202, corresponding, in this example, to the time point at which the subject starts to exhale. In the example shown in FIG. 2A, the humidity of the fluid begins to plateau at a time point approximately halfway between the times indicated by lines 202 and 204. The humidity at the time point represented by line 204 may correspond to the time point during the exhalation at which the humidity of the fluid reaches a defined humidity level (e.g., 44 mg/l, or the like). The defined humidity level may refer to an absolute humidity of the fluid at a defined temperature. For example, the defined humidity level of the fluid may be 44 mg/l H₂O at 37° C. In some examples, the defined humidity level of the fluid may be 30 mg/l H₂O at 37° C., in the range 30 to 44 mg/l H₂O at 37° C., or the like. The defined humidity level may be a point at which the humidity starts to plateau, the point at which the humidity reaches a pre-set value (e.g., a peak value), or the like. The defined humidity level may be indicative of the subject's ISB.

FIG. 2B is a graph showing an example of how flow rate of a fluid may change as a function of time during inhalation and exhalation of a subject, as represented by line 206. The x-axis represents time and the y-axis represents flow rate. In this example, the flow rate of the fluid shown in FIG. 2B corresponds to the humidity of the fluid shown in FIG. 2A (e.g., the humidity and the flow rate correspond to the same fluid). A positive flow rate corresponds to an inhalation whereas a negative flow rate corresponds to an exhalation. As can be seen in FIG. 2B, the flow rate is positive up to a time point indicated by line 202, corresponding to a subject breathing in. The corresponding humidity of the inhaled fluid (e.g., ambient air, oxygen, or the like) remains at a constant level. Between the time points indicated by lines 202 and 204, the flow rate is negative, corresponding to the subject breathing out. The corresponding humidity of the exhaled fluid between time points 202 and 204 increases, which has been humidified by the subject's airways. The subject continues to breath out until there is no fluid left to exhale, at which point the flow rate falls to zero. The area 208 under the curve 206 between times points 202 and 204 represents the volume of fluid exhaled between these time points, and may be indicative of the volume of fluid above the subject's ISB (e.g., 100 ml). The volume of fluid above the subject's ISB may be indicative of a physiological position of the ISB.

An unexpected drop in the humidity may be indicative of the subject having an open mouth. Deviation from a normal, or baseline, ISB may be indicative of a disease state and/or disease progression of the subject (e.g., dysfunctional mucus, or abnormal mucous levels, could shift the ISB away from a normal value). Thus, by determining the ISB before a therapy session (e.g., before the subject received high flow nasal therapy), it may be possible to track any changes in the subject's ISB and respond accordingly.

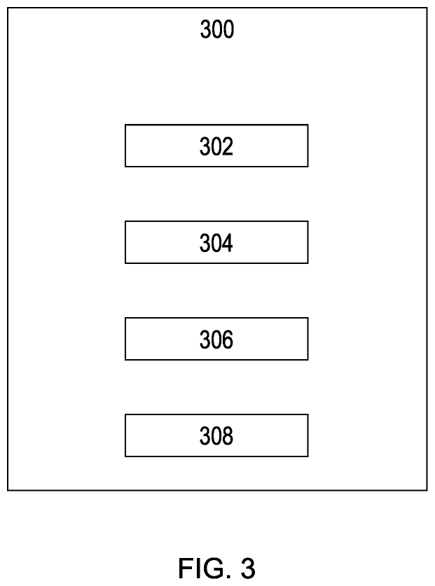

According to a first aspect, an apparatus (e.g., a cannula) for use in high flow nasal therapy is provided. FIG. 3 is a schematic illustration of an example of a cannula 300 for use in high flow nasal therapy. The cannula 300 comprises a first tube 302 for directing a first fluid from a nasal cavity of a subject to a location outside of the subject, and a second tube 304 for directing a second fluid from a supply of the second fluid to the nasal cavity of the subject. In some examples, the first tube 302 may be located in a first nostril of the subject and the second tube 304 may be located in a second nostril of the subject. In other examples, the first tube 302 and the second tube 304 may be located in the same nostril of the subject. In some examples, a first end of the first tube 302 and a second end of the first tube may both be located within a nostril of a subject such that the first fluid enters the first tube within, and exits the first tube into, the nostril of the subject. In this example, the flow rate of the first fluid and/or any momentum of the first fluid may be sufficient to propel it to a location outside of the nostril of the subject. In other examples, the first end of the first tube 302 may be located within a nostril of the subject and the second end of the first tube may be located outside of the subject such that the first fluid exits the first tube at a location outside of the subject. In some examples, the first tube 302 and the second tube 304 are positioned adjacent to one another (e.g., parallel to one another) while, in other examples, the first tube and the second tube are positioned concentrically relative to one another (e.g., one is located inside of the other). In some examples, the first tube 302 may be located within the second tube 304. In other examples, the second tube may be located within the first tube.

The cannula 300 further comprises a flow sensor 306 located within the first tube 302, the flow sensor configured to measure a flow rate of the first fluid moving through the first tube. The flow sensor 306 may comprise a thin-film thermal flow sensor (e.g., a miniaturised thin-film thermal flow sensor). A thin-film thermal flow sensor may include no moving parts. The cannula 300 further comprises a humidity sensor 308 located within the first tube 302, the humidity sensor 308 configured to measure a humidity of the first fluid moving through the first tube. The humidity sensor 308 may comprise an integrated capacitive membrane sensor. The integrated capacitive membrane sensor may be able to measure humidity on the order of microseconds. The flow rate sensor and/or the humidity sensor may be small, robust and/or lightweight. The measured flow rate and the measured humidity are to be used by a processor to control a humidifier to adjust a humidity of the second fluid to be supplied to the subject. The humidifier may be an evaporative humidifier (e.g., that uses a wicking filter), an aerosol humidifier, a passover humidifier, or the like. The processor may be configured to perform one or more steps of the method described herein. The cannula 300 may comprise a transmission unit to send data (e.g., the flow rate data and/or the humidity data) to a processor (e.g., the processor used to control a humidifier). In some examples, the cannula 300 may itself comprise a processor (e.g., the processor used to control a humidifier). In other examples, the processor may be located in the humidifier, a computer, a server, or the like.

In some examples, the first tube 302 and the second tube 304 may be arranged concentrically relative to one another. In some examples, the second tube 304 may be located within the first tube 302. The flow sensor 306 and the humidity sensor 308 may, in some examples, be located between the second tube 304 and the first tube 302. In other examples, the flow sensor 306 and/or the humidity sensor may be located within the first tube, wherein the first tube is located within the second tube. In some examples, the flow sensor 306 and/or the humidity sensor may be located within the second tube, wherein the second tube is located within the first tube. When the two tubes are arranged in this way (e.g., the first tube located within the second tube), a ring-shaped space or cavity may be formed between the first tube and the second tube, in which the flow rate sensor and/or the humidity sensor may be positioned. A benefit of placing the flow rate sensor and/or the humidity sensor between the first tube and the second tube (or within the first tube when the first tube is located within the second tube, or within the second tube when the second tube is located within the first tube) is that the sensors are not in direct contact with tissue (e.g., tissue of the nose of a subject), which may thereby prevent any potential measurement inaccuracies or biases due to movement of the cannula within the subject's tissue. A further benefit of placing the flow rate sensor and/or the humidity sensor between the first tube and the second tube is that a defined channel may be provided in which a fluid (e.g., the first fluid) travels through a tube. In some examples, the tubes may comprise a circular cross-section. In this case, the diameters of the first tube and/or the second tube may be fixed. As a result, a more accurate flow rate (e.g., a volumetric flow rate) may be determined (e.g., based on the speed with which the fluid moves through the first tube and/or the second tube). In other examples, the first tube and/or the second tube may comprise a non-circular cross-section (e.g., oval, hexagonal, square, or the like). In this case, the flow rate of fluid moving through the first and/or second tube may be determined using the cross-sectional area of the first tube and/or the second tube.

In some examples, the cannula may further comprise a humidification connection to couple the cannula to a humidifier configured to adjust a humidity of the second fluid to be supplied to the subject. The cannula and the humidifier may, for example, form part of a system. In other examples, the humidifier, or a part thereof, may form part of the cannula itself.

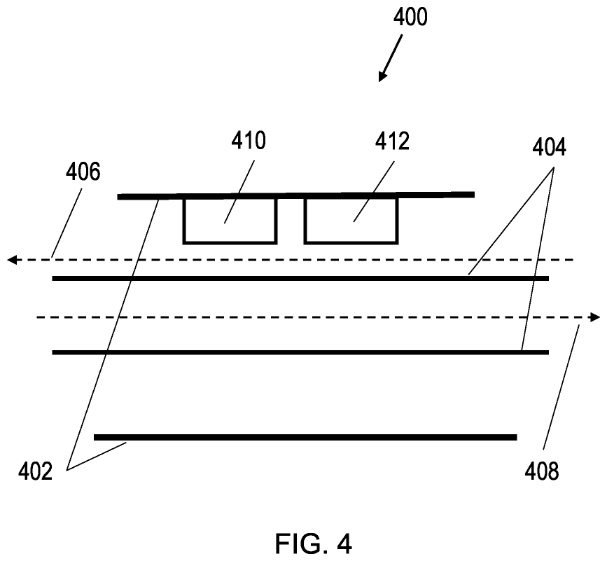

FIG. 4 is an illustration of an example of a cannula 400 for use in high flow nasal therapy. The cannula 400 may comprise or be similar to the cannula 300 discussed above. The example cannula 400 shown in FIG. 4 comprises a first tube 402. The first tube 402 may be suitable or configured for directing a first fluid from a nasal cavity of a subject to a location outside of the subject. The cannula 400 may comprise a second tube 404. The second tube 404 may be suitable for directing a second fluid from a supply of the second fluid to the nasal cavity of the subject. The first fluid may comprise exhaled breath (e.g., exhaled gas (e.g., oxygen, carbon dioxide, or the like), water, saliva, or the like) and/or the second fluid delivered to the subject. For example, when the subject is breathing in, the first fluid (e.g., the fluid moving through the first tube in a direction from the subject's nasal cavity to a location outside of the subject) may include any excess second fluid (e.g. superfluous to the subject's requirements), whereas when the subject is breathing out, the first fluid may include the second fluid and the exhaled breath of the subject. In other words, when the subject is breathing out, the second fluid may still be being supplied to the subject (e.g. the second fluid may be being delivered to the subject via the second tube), which may be vented via the first tube along with the first fluid (e.g. including exhaled breath of the subject). Arrow 406 represents the general direction of movement of the first fluid through the first tube 402 and arrow 408 represents the general direction of movement of the second fluid through the second tube 404. The cannula 400 further comprises a flow rate sensor 410 and a humidity sensor 412. The flow rate sensor 410 and the humidity sensor 412 may be configured to measure a flow rate and a humidity of the first fluid, respectively.

In some examples, the first tube 302, 402 may be suitable for directing a second fluid from a supply of the second fluid to the nasal cavity of the subject and the second tube 304, 404 may be suitable for directing a second fluid from a supply of the second fluid to the nasal cavity of the subject (not shown). In this case, when a subject breathes in, ambient air may enter the subject via the first tube 302, 402. In some examples, the flow rate sensor 306, 410 and/or the humidity sensor 308, 412 may be located in the second tube 304, 404. In some examples, a flow rate sensor 306, 410 may be located in each of the first tube 302, 402 and the second tube 304, 404. In some examples, a humidity sensor 308, 412 may be located in each of the first tube 302, 402 and the second tube 304, 404.

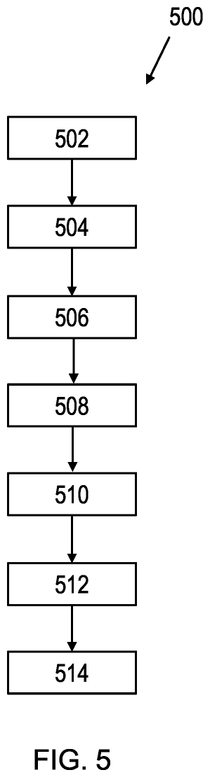

According to a second aspect, a method is provided. FIG. 5 is a flowchart of an example of a method 500 (e.g., a computer-implemented method) for generating a signal to control a humidification setting of a humidifier. The method 500 comprises, at step 502, receiving flow rate data, measured using a flow sensor, the flow rate data indicative of a flow rate of a first fluid moving through a tube for directing the first fluid from a nasal cavity of a subject to a location outside of the subject. The method 500 further comprises, at step 504, receiving humidity data indicative of a humidity of the first fluid moving through the tube. In some examples, the flow rate data and/or the humidity data may be received from the flow rate sensor and the humidity sensor, respectively. In other examples, the flow rate data and/or the humidity data may be received from a processor, a storage medium (e.g., a hard drive, the cloud, or the like), or the like. For example, the flow rate data and/or the humidity data may have been acquired previously using a flow rate sensor and a humidity sensor, respectively, and stored in the storage medium.

The method 500 further comprises, at step 506, determining, based on the flow rate data and/or the humidity data, a first time point at which the subject begins an exhalation. As explained with respect to FIG. 2, a first time point at which the subject begins an exhalation may be determined based on the flow rate data (e.g., when the flow rate becomes negative), the humidity data (e.g., when the humidity begins to increase), or a combination of the two.

The method further comprises, at step 508, determining, based on the humidity data, a second time point during the exhalation at which the humidity of the first fluid reaches a defined humidity level. As explained with respect to FIG. 2, the defined humidity level may comprise a point at which the humidity starts to plateau, the point at which the humidity reaches a pre-set value (e.g., a peak value), or the like. In some examples, the defined humidity level may be set (e.g., pre-set) to a value of 30 mg/l H₂O at 37° C., 40 mg/l H₂O at 37° C., 44 mg/l H₂O at 37° C., or the like.

The method further comprises, at step 510, determining, based on the flow rate data, a volume of the first fluid passing the flow sensor from the first time point to the second time point. In some examples, the volume of the first fluid passing the flow sensor may be determined by calculating the volume of the first fluid passing through a cross-section of the tube from the first time point to the second time point. The volume of fluid may be determined by integrating the flow rate of the fluid passing the flow sensor between the first time point and the second time point.

The method further comprises, at step 512, comparing the volume of the first fluid to a reference volume. The reference volume may be indicative of a subject's natural ISB (e.g., the subject's ISB during rest and without being supplied a source of fluid via high flow nasal therapy or otherwise). If the volume of fluid above the subject's ISB increases (e.g., increases above the subject's natural volume), this may be a sign that the subject's airways are drying out, such that the subject may benefit from a supply of fluid (e.g., the second fluid) with a relatively higher humidity level than the fluid that they are currently breathing in (e.g., the second fluid). If the volume of fluid above the subject's ISB decreases (e.g., decreases below the subject's natural volume), this may be a sign that the subject's airways are too humid (e.g., the subject is receiving a fluid with a higher humidity than needed to humidify the fluid that they are breathing in, in order to prevent their airways drying out), such that the subject may benefit from a supply of fluid with a relatively lower humidity level.

The method further comprises, at step 514, generating, based on the comparison, a signal to control a humidification setting of a humidifier such that the humidity of a second fluid to be supplied to the subject is updated. For example, if it is determined at step 512 that the subject's ISB has increased, then the signal may comprise an instruction to increase the humidity setting of the humidifier such that the fluid supplied to the subject (e.g., the second fluid) is at a relatively higher humidity.

As the subject breathes out, the first fluid passing through the first tube (e.g., the fluid exiting the subject) may comprise both the exhaled breath of the subject and the second fluid. In this case, the second fluid will mix with the fluid exhaled by the subject, which may thereby change the properties of the exhaled fluid (e.g., the flow rate and/or the humidity of the exhaled fluid). For example, the second fluid may lower, or dilute, the water content, and thus lower the humidity level, of the exhaled fluid, which may impact the determination of the subject's ISB. Steps 602, 604, 606 and 608 of method 600, described below, aim to overcome this issue.

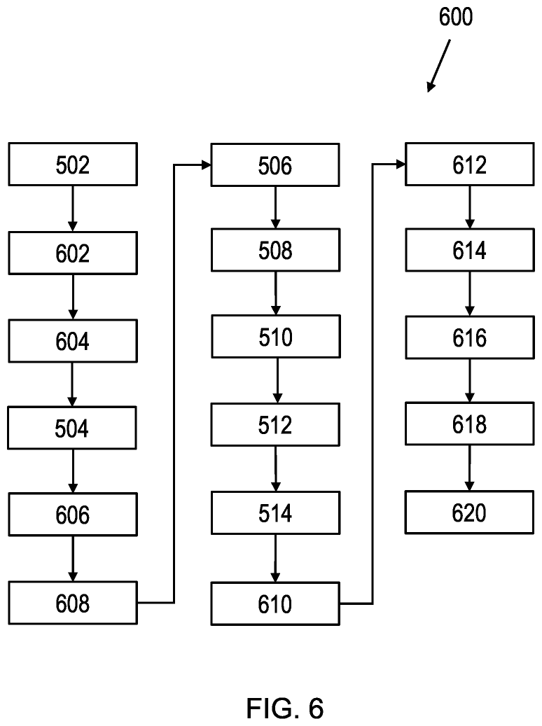

FIG. 6 is a flowchart of an example of a method 600 (e.g., a computer-implemented method) for generating a signal to control a humidification setting of a humidifier. The method 600 comprises, at step 602, determining, at a third time point, a reference flow rate of the second fluid. The reference flow rate may be referred to as a base flow rate, a baseline flow rate, or the like. In other words, the reference flow rate of the second fluid may refer to the flow rate of the second fluid to be supplied, or being supplied, to the subject. The method 600 further comprises, at step 604, applying, based on the reference flow rate, a correction to the flow rate data to account for the flow rate of the second fluid. In some examples, the correction may comprise subtracting the flow rate of the second fluid from the flow rate of the first fluid, wherein the first fluid may comprise fluid exhaled from the subject's lungs and the second fluid.

The method 600 further comprises, at step 606, determining, at a third time point, a reference humidity of the second fluid. The reference humidity may be referred to as a base humidity, a baseline humidity, or the like. In other words, the reference humidity of the second fluid may refer to the humidity of the second fluid to be supplied, or being supplied, to the subject. The method 600 further comprises, at step 608, applying, based on the reference humidity, a correction to the humidity data to account for the humidity of the second fluid to be supplied to the subject. In some examples, the third time point may be a time between inhalation and exhalation of the subject (e.g., where no breathing occurs). The reference flow rate and/or the reference humidity may be used to correct the flow rate data and/or the humidity data, respectively, when the subject starts to exhale. The reference flow rate and/or the reference humidity may be updated periodically (e.g., every breath, every other breath, every 10 breaths, every minute, every 30 minutes, or the like).

The humidity of the first fluid corrected for the humidity of the second fluid may be determined using the following equation:

$$He ( t ) = \frac{H t o t ( t ) - H b}{( \frac{F t o t ( t ) - F b}{F t o t ( t )} )} + H b ,$$

where He(t) is the humidity of the first fluid corrected for the humidity of the second fluid at time t, Htot(t) is the measured humidity (e.g., measured using the humidity sensor) of the first fluid (e.g., the fluid exiting the subject during an exhalation including an exhaled portion and the second fluid) at time t, Hb is the reference humidity, Ftot(t) is the flow rate of the first fluid (e.g., the fluid exiting the subject during an exhalation including an exhaled portion and the second fluid) at time t, and Fb is the reference flow rate.

The volume of the first fluid, corrected for the flow rate of the second fluid, passing the flow sensor from the first time point to the second time point may be calculated using the following equation:

∫₀^{t}Ftot(t)−Fb,

where the flow rate of the first fluid may be integrated over a period of time to obtain a volume. For example, the flow rate of the first fluid, corrected for the flow rate of the second fluid, may be integrated from a time in which the subject begins to exhale until the time point in which humidity of the first fluid, corrected for the flow rate of the second fluid, reaches a defined humidity level (e.g., 44 mg/l).

The method 600 further comprises, at step 610, determining, based on the flow rate data and/or the humidity data, a fourth time point at which the subject begins to inhale. With reference to FIG. 2, the time point at which the subject begins to inhale may be determined using the flow rate data (e.g., by determined when the flow rate becomes positive), the humidity data (e.g., when there is a sudden drop in the measured humidity), or a combination of both. A drop in humidity may be indicative of a subject beginning to inhale, because the humidity sensor may measure a relatively high humidity level corresponding to the first fluid as the subject breathes out, which may be followed by a relatively low humidity level as the subject begins to breath in because the humidity sensor may be measuring the humidity of an excess flow of the second fluid supplied to the subject. The method 600 further comprises, at step 612, generating a signal to control a flow rate of the second fluid, such that the second fluid to be supplied to the subject between the first time point and the fourth time point is reduced. Controlling the flow rate of the second fluid may conserve energy and/or resources by reducing (e.g., lowering the flow rate of, or turning off) a supply of the second fluid to the subject as the subject exhales.

The method 600 further comprises, at step 614, receiving a user preference to reduce or increase a level of humidity in the second fluid to be supplied to the subject. This may be of benefit if, for example, the subject is uncomfortable with the level of humidity of the supplied fluid (e.g., the second fluid) that is being used to keep their ISB stable. In some examples, the subject may be able to input a user preference to change a setpoint ISB volume value, for example from 100 to 90, to provide a higher, or a lower, humidity. The method 600 further comprises, at step 616, updating, based on the user preference, the generated signal to control a humidification setting of a humidifier.

The method 600 further comprises, at step 618, generating an alert signal in response to determining that oscillations in the flow rate data exceed a defined frequency and/or in response to determining that the humidity of the first fluid falls below a threshold level. Oscillations in the flow rate data may be indicative of water, or condensation, within the first tube and/or the second tube. The humidity falling below a threshold value may be indicative of the subject breathing through their mouth rather than their nose, or due to onset, or progression, of a disease. In some examples, an alert signal is generated in response to determining that the humidity of the first fluid falls below a threshold level for a defined amount of time (e.g., 30 seconds, 1 minute, 2 minutes, or the like). This may differentiate between the humidity falling due to the subject beginning to inhale and due to, for example, the subject breathing through their mouth.

The method 600 further comprises, at step 620, generating an alert signal in response to determining that the volume of the first fluid deviates from the reference volume by a defined amount. The deviation of the volume of the first fluid from the reference volume may similarly be indicative of the subject breathing through their mouth rather than their nose, or due to onset, or progression, of a disease.

In some examples, the subject may receive a supply of the second fluid via a second tube. The reference volume may be determined based on data obtained in the absence of the second fluid being supplied to the subject via the second tube. The reference volume may be indicative of a default state of a subject. A default state of a subject may, for example, refer to a subject who is not receiving a supply of fluid (e.g., via high flow nasal therapy).

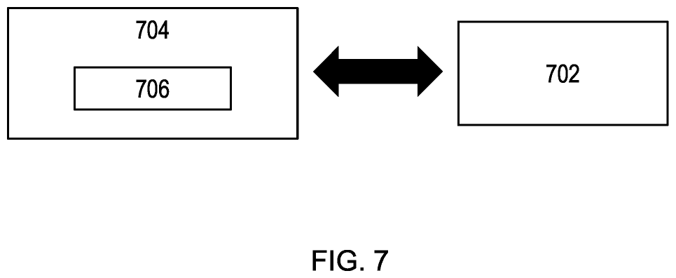

According to a third aspect, a computer program product is provided. FIG. 7 is a schematic illustration of an example of a processor 702 in communication with a computer-readable medium 704. According to various embodiments, a computer program product comprises a non-transitory computer readable medium 704, the computer readable medium having computer readable code 706 embodied therein, the computer readable code being configured such that, on execution by a suitable computer or processor 702, the computer or processor is caused to perform one or more steps of the methods 500, 600 discussed herein.

The present disclosure also provides an apparatus for use in high flow nasal therapy. The apparatus may comprise a flow sensor configured to measure a flow rate of a first fluid moving through a tube, the first fluid moving in a direction from a nasal cavity of a subject to a location outside of the subject. The apparatus may further comprise a humidity sensor configured to measure a humidity of the first fluid moving through the tube from the nasal cavity of the subject to the location outside of the subject. The measured flow rate and the measured humidity may be used by a processor to control a humidifier to adjust a humidity of a second fluid to be supplied to the subject. The processor may be configured to perform one or more steps of the methods described herein. In some examples, the flow sensor of the apparatus may comprise a thin-film thermal flow sensor. In some examples, the humidity sensor of the apparatus may comprise an integrated capacitive membrane sensor.

The present disclosure also provides a system, such as a system for use in high flow nasal therapy. The system may comprise a first tube for directing a first fluid from a nasal cavity of a subject to a location outside of the subject, and a second tube for directing a second fluid from a supply of the second fluid to the nasal cavity of the subject. The system may comprise a flow sensor located within the first tube, the flow sensor configured to measure a flow rate of the first fluid moving through the first tube. The system may comprise a humidity sensor located within the first tube, the humidity sensor configured to measure a humidity of the first fluid moving through the first tube. The system may comprise a processor to control a humidifier to adjust a humidity of the second fluid to be supplied to the subject. The processor may be configured to perform one or more steps of the method described herein. In some examples, the system may further comprise a humidifier configured to adjust a humidity of the second fluid to be supplied to the subject. In some examples, the flow sensor of the system may comprise a thin-film thermal flow sensor. In some examples, the humidity sensor of the system may comprise an integrated capacitive membrane sensor. In some examples, the system may comprise a humidifier configured to adjust a humidity of the second fluid to be supplied to the subject.

The embodiments discussed herein may be implemented in a ventilator (e.g., for blower based HFNT), as a standalone unit attached to a blender based HFNT unit, or the like.

The processor 702 can comprise one or more processors, processing units, multi-core processors or modules that are configured or programmed to control components of the cannula/system in the manner described herein. In particular implementations, the processor 702 can comprise a plurality of software and/or hardware modules that are each configured to perform, or are for performing, individual or multiple steps of the method described herein.

The term “module”, as used herein is intended to include a hardware component, such as a processor or a component of a processor configured to perform a particular function, or a software component, such as a set of instruction data that has a particular function when executed by a processor.

It will be appreciated that the embodiments of the invention also apply to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to embodiments of the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g., Java interpreter instructions). Alternatively, one or more or all of the sub-routines may be stored in at least one external library file and linked with a main program either statically or dynamically, e.g., at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

Variations to the disclosed embodiments can be understood and effected by those skilled in the art in practicing the principles and techniques described herein, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored or distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A cannula for use in high flow nasal therapy, comprising:
a first tube for directing a first fluid from a nasal cavity of a subject to a location outside of the subject;
a second tube for directing a second fluid from a supply of the second fluid to the nasal cavity of the subject;
a flow sensor located within the first tube, the flow sensor configured to measure a flow rate of the first fluid moving through the first tube; and
a humidity sensor located within the first tube, the humidity sensor configured to measure a humidity of the first fluid moving through the first tube;
wherein the measured flow rate and the measured humidity are to be used by a processor to control a humidifier to adjust a humidity of the second fluid to be supplied to the subject.

2. A cannula according to claim 1, wherein the first tube and the second tube are arranged concentrically relative to one another and/or wherein the second tube is located within the first tube, and wherein the flow sensor and the humidity sensor are located between the second tube and the first tube.

3. A cannula according to claim 1, wherein the flow sensor comprises a thin-film thermal flow sensor.

4. A cannula according to claim 1, wherein the humidity sensor comprises an integrated capacitive membrane sensor.

5. A cannula according to claim 1, further comprising:
a humidification connection to couple the cannula to a humidifier configured to adjust a humidity of the second fluid to be supplied to the subject.

6. A computer-implemented method comprising:
receiving flow rate data, measured using a flow sensor, the flow rate data indicative of a flow rate of a first fluid moving through a tube for directing the first fluid from a nasal cavity of a subject to a location outside of the subject;
receiving humidity data indicative of a humidity of the first fluid moving through the tube;
determining, based on the flow rate data and/or the humidity data, a first time point at which the subject begins an exhalation;
determining, based on the humidity data, a second time point during the exhalation at which the humidity of the first fluid reaches a defined humidity level;
determining, based on the flow rate data, a volume of the first fluid passing the flow sensor from the first time point to the second time point;
comparing the volume of the first fluid to a reference volume; and
generating, based on the comparison, a signal to control a humidification setting of a humidifier such that the humidity of a second fluid to be supplied to the subject is updated.

7. The computer-implemented method of claim 6, further comprising:
determining, at a third time point, a reference flow rate of the second fluid; and
applying, based on the reference flow rate, a correction to the flow rate data to account for the flow rate of the second fluid.

8. The computer-implemented method of claim 6, further comprising:
determining, at a third time point, a reference humidity of the second fluid; and
applying, based on the reference humidity, a correction to the humidity data to account for the humidity of the second fluid to be supplied to the subject.

9. The computer-implemented method of claim 6, further comprising:
determining, based on the flow rate data and/or the humidity data, a fourth time point at which the subject begins to inhale; and
generating a signal to control a flow rate of the second fluid, such that the second fluid to be supplied to the subject between the first time point and the fourth time point is reduced.

10. The computer-implemented method of claim 6, further comprising:
receiving a user preference to reduce or increase a level of humidity in the second fluid to be supplied to the subject; and
updating, based on the user preference, the generated signal to control a humidification setting of a humidifier.

11. The computer-implemented method of claim 6, further comprising:
generating an alert signal in response to determining that oscillations in the flow rate data exceed a defined frequency and/or in response to determining that the humidity of the first fluid falls below a threshold level.

12. The computer-implemented method of claim 6, wherein the subject is to receive a supply of the second fluid via a second tube, and wherein the reference volume is determined based on data obtained in the absence of the second fluid being supplied to the subject via the second tube.

13. The computer-implemented method of claim 12, further comprising:
generating an alert signal in response to determining that the volume of the first fluid deviates from the reference volume by a defined amount.

14. The computer-implemented method of claim 6, wherein the defined humidity level is in the range 30 to 44 mg/l H₂O at 37° C.

15. A computer program product comprising a non-transitory computer readable medium, the computer readable medium having computer readable code embodied therein, the computer readable code being configured such that, on execution by a suitable computer or processor, the computer or processor is caused to perform the method of claim 6.
